(19) United States
(12) Patent Application Publication
(10) Pub. No.: US 2024/0402151 A1
(43) Pub. Date: Dec. 5, 2024

(54) FLUIDIC TUBING ASSEMBLY FOR BLOOD ANALYZER

(71) Applicant: Siemens Healthcare Diagnostics Inc., Tarrytown, NY (US)

(72) Inventor: Christian Pudduck, Norfolk, MA (US)

(73) Assignee: Siemens Healthcare Diagnostics Inc., Tarrytown, NY (US)

(21) Appl. No.: 18/701,500

(22) PCT Filed: Oct. 17, 2022
(86) PCT No.: PCT/US2022/078190

§ 371 Date: Apr. 15, 2024

Related U.S. Application Data
(60) Provisional application No. 63/270,206, filed on Oct. 21, 2021.

(51) Int. Cl. G01N 33/49; A61M 39/10

(52) U.S. Cl. CPC G01N 33/4915; A61M 39/1011

(57) ABSTRACT

A fluidic tubing assembly and method for a blood analyzer comprising a base, a first tube, and a second tube. The base is connectable to the blood analyzer and has a front side, a rear side, a first side, a second side, a top side, and a bottom side. A first connector is supported by the first side. The first end of the first tube is connected to the first connector. A second connector is supported by the top side. The second end of the first tube is connected to the second connector. A third connector is supported by the top side. The first end of the second tube is connected to the third connector. A fourth connector is supported by the bottom side. The second end of the second tube is connected to the fourth connector.

This application claims benefit under 35 USC § 119(e) of U.S. Provisional Application No. 63/270,206, filed Oct. 21, 2021. The entire contents of the above-referenced patent application are hereby expressly incorporated herein by reference.

### BACKGROUND

Modern-day blood analyzers are designed to use a small volume of a patient's blood for measurement. To achieve this, the analyzers employ fluid lines with small bores to transport the blood from a sampling device to sensors and ultimately to a waste container. Because the fluid lines are small, they are susceptible to blockage due to blood clots, protein build-up, and salt crystal growth. Blockage of the fluid lines leads to a loss of functionality of the analyzer.

Liquid wash solution containing anti-microbial agent and surfactants can be cycled through the fluid lines. However, this does not fully prevent biofilm and protein growth over repeated samples. Washing cannot always remove blood clots or other particulates.

Another way of trying to prevent blockages is to replace all the wetted components routinely or when blockages do occur. This generally requires a service technician to visit the site of the analyzer, which takes time and places the analyzer out of use a problem.

Accordingly, a need exists for an improved fluidic tubing assembly in which the functional parts of the assembly are formed as a modular unit and are quickly and easily replaceable by the user if blockage occurs. It is to such a fluidic tubing assembly that the inventive concepts disclosed herein are directed.

### SUMMARY OF THE INVENTIVE CONCEPTS

The inventive concepts disclosed and claimed herein generally relate to a fluidic tubing assembly for a blood analyzer having a housing supporting a fluid sample assembly, a sensor assembly, and a fluid waste assembly. The fluidic tubing assembly includes a base positionable in the housing of the blood analyzer; a plurality of connectors extending from the base so each of the connectors is removably connectable to at least one of the fluid sample assembly, the sensor assembly, and the fluid waste assembly; and a plurality of tubes where each of the tubes extends from one of the connectors to another one of the connectors to establish fluid communication from one of the connectors to the other connector, wherein fluid communication is established between the fluid sample assembly, the sensor assembly, and the fluid waste assembly through the fluidic tubing assembly when the fluidic tubing assembly is positioned in the housing.

In another aspect, the fluidic tubing assembly includes a base, a first tube, a second tube, a first connector, a second connector, a third connector, and a fourth connector. The base is connectable to the blood analyzer and has a front side, a rear side opposite the front side, a first side, a second side opposite the first side, a top side, and a bottom side opposite the top side. The first tube has a first end and a second end. The second tube has a first end and a second end. The first connector is supported by the first side of the base and defines a first fluid inlet. The first end of the first tube is connected to the first connector. The second connector is supported by the top side of the base and defines a first fluid outlet. The second end of the first tube is connected to the second connector. The third connector is supported by the top side of the base and defines a second fluid inlet. The first end of the second tube is connected to the third connector. The fourth connector is supported by the bottom side of the base and defines a second fluid outlet. The second end of the second tube is connected to the fourth connector.

### DESCRIPTION OF THE DRAWINGS

To assist those of ordinary skill in the relevant art in making and using the inventive concepts disclosed herein, reference is made to the appended drawings and schematics, which are not intended to be drawn to scale, and in which like reference numerals are intended to refer to the same or similar elements for consistency. For purposes of clarity, not every component may be labeled in every drawing. Certain features and certain views of the figures may be shown exaggerated and not to scale or in schematic in the interest of clarity and conciseness. In the drawings:

FIG. 1 is a perspective view of a blood analyzer with a fluidic tubing assembly constructed in accordance with the inventive concepts disclosed herein.

FIG. 2 is an exploded, perspective view of the blood analyzer of FIG. 1 showing the fluidic tubing assembly removed from the blood analyzer.

FIG. 3 a front perspective view of the fluidic tubing assembly.

FIG. 4 is an exploded, perspective view of the fluidic tubing assembly of FIG. 3.

FIG. 5 is a rear, transparent perspective view of the fluidic tubing assembly.

FIG. 6 is a sectional view taken along line 6-6 of FIG. 3.

FIG. 7 is a sectional view taken along line 7-7 of FIG. 3.

FIG. 8 is a sectional view taken along line 8-8 of FIG. 3.

FIG. 9 is a front perspective view of another embodiment of a fluidic tubing assembly constructed in accordance with the inventive concepts disclosed herein.

FIG. 10 is a bottom plan view of the fluidic tubing assembly of claim 9.

FIG. 11 is a front elevational view of a CO-oximetry optical cell used with the fluidic tubing assembly of FIG. 9.

FIG. 12 is a sectional view taken along line 12-12 of FIG. 11.

### DETAILED DESCRIPTION OF EXEMPLARY EMBODIMENTS

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. The language used herein is intended to be given the broadest possible scope and meaning, and the embodiments are meant to be exemplary-not exhaustive. Also, it is to be understood that the phraseology and terminology employed herein is for the purpose of description and should not be regarded as limiting.

Unless otherwise defined, scientific and technical terms used in connection with the presently disclosed and claimed inventive concept(s) shall have the meanings commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All the articles, compositions and/or methods disclosed and claimed herein can be made and executed without undue experimentation, given the present disclosure. While the articles, compositions and methods of the inventive concept(s) have been described in terms of preferred embodiments, it will be apparent to those of skill in the art that variations may be applied to the articles, compositions and/or methods and in the steps or in the sequence of steps of the methods described herein without departing from the concept, spirit and scope of the inventive concept(s). All such similar substitutes and modifications apparent to those skilled in the art are deemed to be within the spirit, scope and concept of the inventive concept(s) as defined by the appended claims.

As utilized under the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “sample” and variations thereof is intended to include biological tissues, biological fluids, chemical fluids, chemical substances, suspensions, solutions, slurries, mixtures, agglomerations, tinctures, slides, powders, or other preparations of biological tissues or fluids, synthetic analogs to biological tissues or fluids, bacterial cells (prokaryotic or eukaryotic), viruses, single-celled organisms, lysed biological cells, fixed biological cells, fixed biological tissues, cell cultures, tissue cultures, genetically engineered cells and tissues, genetically engineered organisms, and combinations thereof, for example.

In the following detailed description of embodiments of the inventive concept, numerous specific details are set forth in order to provide a more thorough understanding of the inventive concept. However, it will be apparent to one of ordinary skill in the art that the inventive concept within the disclosure may be practiced without these specific details. In other instances, well-known features have not been described in detail to avoid unnecessarily complicating the instant disclosure.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Described herein, and shown in the accompanying figures, are several non-limiting embodiments of apparatus of the presently claimed and disclosed inventive concepts which may be used in association with collection syringes and fluid sample analyzers for removing bubbles of air or other gases from a fluid sample for analysis by a fluid sample analyzer. The fluid sample is generally from a biological source. A “fluid” refers to any substance that has no fixed shape and yields easily to external pressure.

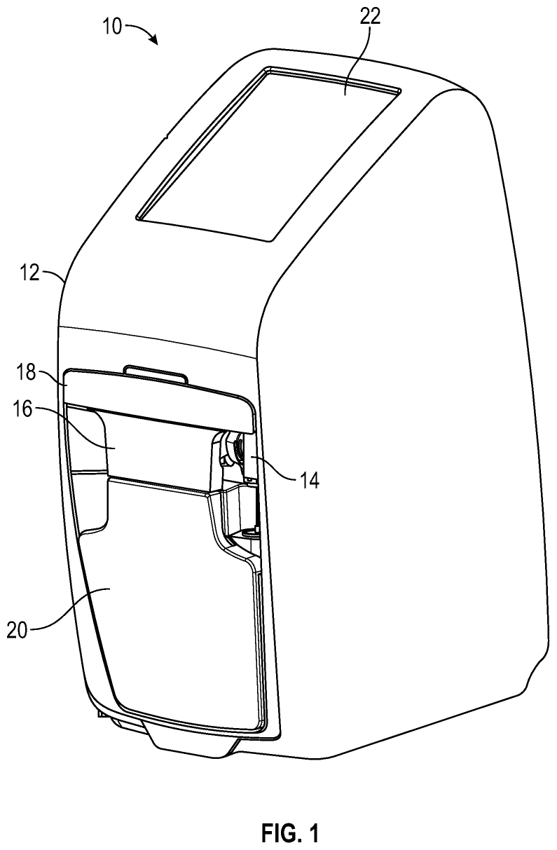

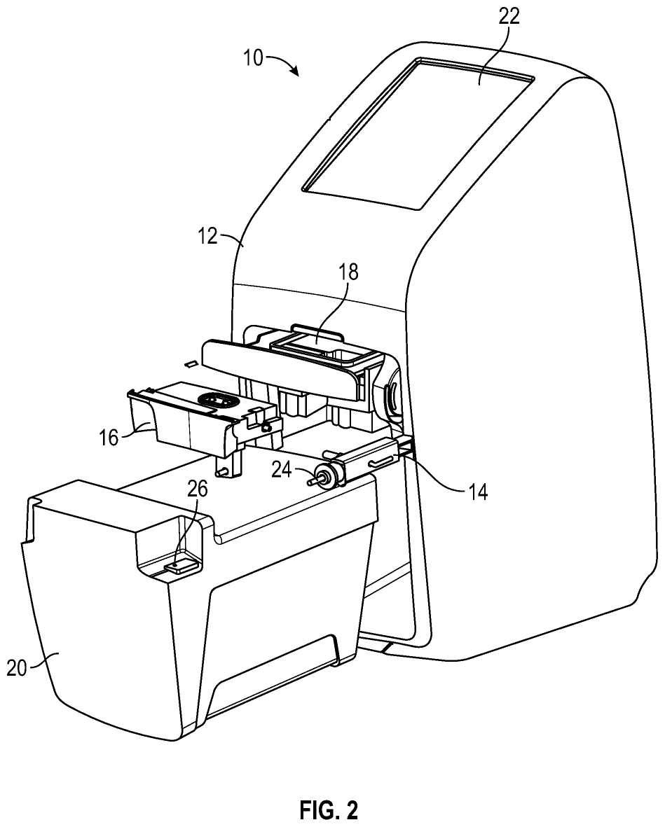

Referring now to the drawings, and more particularly to FIGS. 1 and 2, a blood analyzer 10 for analyzing one or more samples for one or more target analytes is illustrated. In certain embodiments, the blood analyzer 10 is a point of care analyzer or a blood analyzer as known in the art. Exemplary point of care analyzers are available from Siemens Healthcare Diagnostics, Inc. and are sold under the trademarks: RAPIDLab 1200, RapidLab 348EX, RAPIDPoint 500, RAPIDLab 248/348, RAPIDPoint 400/405, and RAPIDPoint 340/350 Systems. Other commercially available point of care instruments are available from Roche Molecular Systems Inc., Medica Corp., Radiometer Medical (Denmark), and Nova Biomedical Corp.

The blood analyzer 10 includes an enclosure 12 for housing and supporting multiple sample analyzing components and/or modules. These components may include a sample receiving assembly 14, a fluidic tubing assembly 16, a sensor assembly 18, and a reagent assembly 20. The enclosure 12 may also support a display screen 16 for illustrating the progress of a test.

The fluid sample to be introduced to the blood analyzer 10 may comprise any biological material taken from a subject, for example, such as a bodily fluid, infection, or abscess collected from the subject by suitable methods and devices known in the art. Bodily fluids include but are not limited to urine, whole blood, blood serum, blood plasma, saliva, cerebrospinal fluid, pleural fluid, dialysate fluid, nasopharyngeal swabs, vaginal swabs, tears, tissues, and the like. The sample may further include any suitable buffers, diluents, or the like as needed or desired for the particular sample. In particular embodiments, the sample comprises a blood sample, which may be: a whole blood sample comprising plasma and whole blood cells; a plasma sample; or a serum sample. In a particular embodiment, the sample comprises a whole blood sample. The whole blood sample may comprise red blood cells, platelets and the like. In other embodiments, the blood sample comprises a plasma sample. To obtain the plasma sample, the sample may have been treated to remove a plurality of the whole blood cells using known methods and components such as centrifugation or commercially available porous membranes.

The sample receiving assembly 14 is adapted for introducing a liquid sample from a transport container (not shown) to the sensor assembly 18 for analysis. An example of a sample receiving assembly 14 is disclosed in U.S. Pat. No. 10,928,409, which is hereby expressly incorporated herein by reference. In one example, the sample receiving assembly 14 includes a sample probe 24 that may be rotatable to selected positions so the sample probe 24 can receive a fluid sample from different types of sample transport containers. Examples of sample transport containers are syringes, vacutainers, and capillary tubes (not shown). The sample probe 24 may also be oriented in a stand-by mode (e.g., vertically) to seal against a fluid outlet 26 of the reagent assembly 20 whereby the sample receiving assembly 12 is used to transport fluid from the reagent assembly 20 to the sensor assembly 18.

The reagent assembly 20 holds a plurality of reagent fluids used in the test. The reagents may be provided in reservoirs, such as sealed bags or bottles (not shown). The reagent assembly 20 may comprise one or several reservoirs pre-filled with process liquids (as known to a person skilled in the art: QC1, QC2, QC3, CRL3 (S1940), CRL2 (S1930), RINSE/CAL1 (S1920)) having a known composition. The skilled person will appreciate that other chemicals may be provided dependent on the exact test required.

The reagent assembly 20 may include a rubber teat (not shown) defining the fluid outlet 26, for example, such that when brought into sealing engagement with sample receiving assembly 14, the reagent assembly 20 is in fluid communication with the sensor assembly 18 enabling reagent fluid to flow from the reagent assembly 20 to the sensor assembly 18. The reagent assembly 20 can be integrated as part of the blood analyzer 10 or may otherwise be configured to be removable/disposable.

The sensor assembly 18 includes sensors (not shown) which are used to contact a fluid sample. Typically, the sensors of the sensor assembly 18 contain sensors comprising rare metal alloys (such as vanadium bronze) and functionalized with membranes holding active ingredients. The sensor assembly 18 may be integrated into the blood analyzer 10 or may otherwise be removable/disposable.

The sensor assembly 18 may be in direct or indirect communication with a computing unit (not shown) which may collect, store, and analyze analytical test results from the sensors according to known methods.

After delivery of a fluid sample to the sensor assembly 18, the blood analyzer 10 may introduce the fluids from the reagent assembly 20 and prepare the blood analyzer 10 for introduction of a subsequent fluid sample.

Following an analysis of a fluid sample, fluid sample and/or expended reagent is transported to a waste fluid collection member (not shown), such as a bag, pouch, or reservoir (not shown). In one embodiment, the waste fluid collection member may be incorporated as part of the reagent assembly 20.

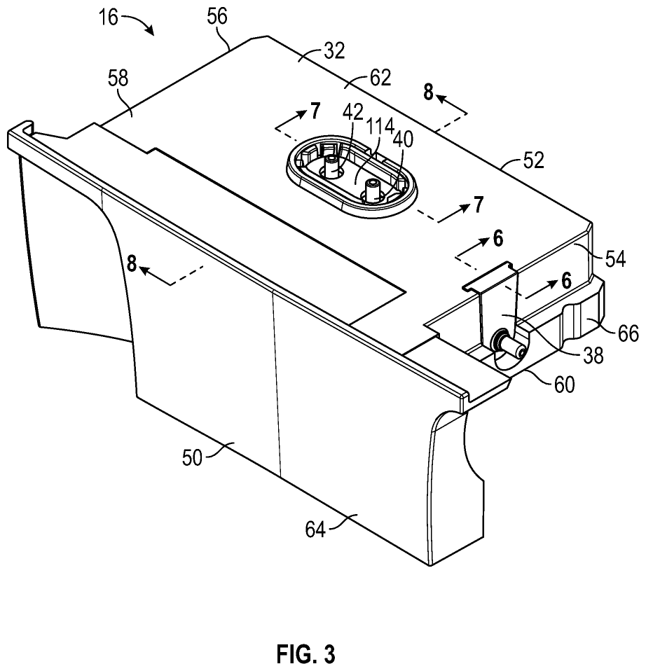

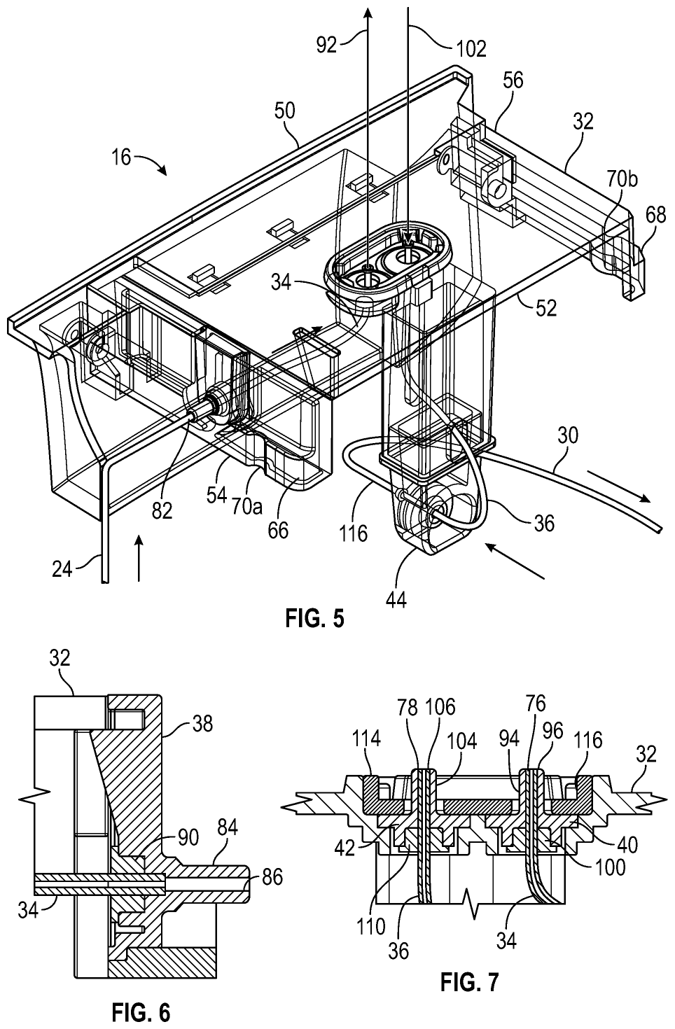

Referring now to FIGS. 3-8, the fluidic tubing assembly 16 is illustrated in more detail. The fluidic tubing assembly 16 is a modular unit that provides fluid communication from the sample receiving assembly 14 to the sensor assembly 18 and from the sensor assembly 18 to a waste conduit 30 (FIG. 5) in fluid communication with the waste fluid collection member. The fluid tubing assembly 16 is configured to be positioned in the enclosure 12 of the blood analyzer 10 and broadly includes a base 32, a first tube 34, a second tube 36, a first connector 38, a second connector 40, a third connector 42, and a fourth connector 44. The fluidic tubing assembly 16 is configured such that the first tube 34 and the second tube 36 connecting the various components of the blood analyzer 10 may be removably disconnected and/or replaced from the blood analyzer 10 without direct contact with the first tube 34 and the second tube 36. Further, the removal of fluidic tubing assembly 16 permits for the simultaneous replacements of all fluidic tubing between the sample receiving assembly 14, the sensor assembly 18, and the reagent assembly 20.

The base 32 supports the others parts of the fluidic tubing assembly 16 so a fluid path is established between the components while allowing the fluidic tubing assembly 16 to be easily replaced. In a non-limiting example, the base 32 has a generally rectangular configuration with a front side 50, a rear side 52 opposite the front side 50, a first side 54, a second side 56 opposite the first side 54, a top side 58, and a bottom side 60 opposite the top side 58. The base 32 may be formed of multiple parts. For example, the base 32 may include two main parts—an interior portion 62 and an exterior portion 64. The interior portion 62 supports the fluidic components of the fluidic tubing assembly 16 and the exterior portion 64 serves as an exterior covering. It should be appreciated that the base 32 may be formed in a variety of ways and with a variety of parts. For example, the base 32 may be formed as a single piece.

Referring to FIG. 5, the first side 54 of the base 32 has a first rail 66 extending from the rear side 52 toward the front side 50, and the second side 56 of the base 32 has a second rail 68 extending from the rear side 52 toward the front side 50. The rails 66 and 68 facilitate insertion and withdrawal of the fluidic tubing assembly 16 into and from the enclosure 12. Also, the rails 66 and 68 may include structures for securing the fluidic tubing assembly 16 within the enclosure. For example, the rails 66 and 68 may include grooves 70a and 70b for receiving retaining pins (not shown) of the blood analyzer 10

Best shown in FIG. 5, the first tube 34 transports fluid from the sample receiving assembly 16 to the sensor assembly 18, and the second tube 36 transports the fluid from the sensor assembly 18 to the waste conduit 30. The first tube 34 has a first end 74 and a second end 76, and the second tube 36 has a first end 78 and a second end 80. The first tube 34 and the second tube 36 may be formed of a suitable flexible, polymeric material. The first tube 34 and the second tube 36 may have an inner diameter in a range of about 0.020 inches to about 0.040 inches. The first tube 34 and the second tube 36 are each illustrated as being a single piece. However, each of the first tube 34 and the second tube 36 may formed of a plurality of pieces, components, or sections that are connected, attached, or otherwise assembled to form a fluid conduit for transporting fluid from the sample receiving assembly 16 to the sensor assembly 18 and from the sensor assembly 18 to the waste conduit 30, respectively.

The first connector 38 is adapted to mate with a fluid outlet 82 of the sample receiving assembly 16 in a way that fluid communication is established between the sample receiving assembly 16 and the first end 74 of the first tube 34 and in a way that the first connector 38 can be quickly disconnected from the fluid outlet 82 of the sample receiving assembly 16.

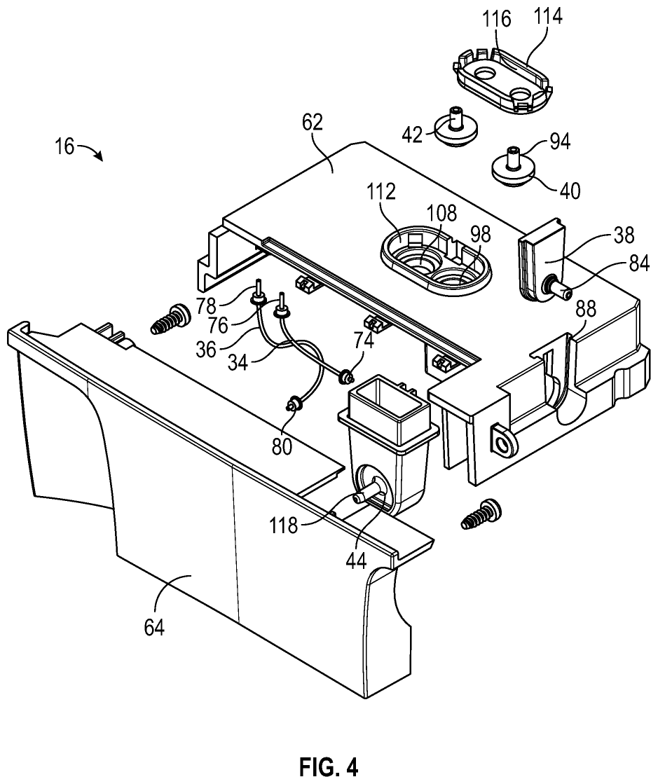

In one embodiment, the first connector 38 has a nipple 84 (FIGS. 4-6) extending away from the base 32 and slidingly mateable with the fluid outlet 82 of the sample receiving assembly 16 (FIG. 5). The nipple 84 has a flow passage 86. The first connector 38 is supported by the first side 54 of the base 32 and defines a first fluid inlet. The first side 54 of the base 32 may have a slot 88 (FIG. 4) configured to slidingly receiving the first connector 38. The first end 74 of the first tube 34 is connected to the first connector 38 to establish fluid communication with the flow passage 86 of the nipple 84. The first connector 38 may also include a seal member 90 (FIG. 6) positioned between the first end 74 of the first tube 34 and the nipple 84 to form a fluid tight seal. It should be appreciated the first connector 38 may be formed in any shape connectable to and disconnectable from the fluid outlet 82 of the sample receiving assembly 16.

The second connector 40 is adapted to mate with a fluid inlet (represented by arrow 92 in FIG. 5) of the sensor assembly 18 in a way that fluid communication is established between the second end 76 of the first tube 34 and the fluid inlet 92 of the sensor assembly 18 and in a way that the second connector 40 can be quickly disconnected from the fluid inlet 92 of the sensor assembly 18.

In one embodiment, the second connector 40 has a nipple 94 (FIGS. 4, 5, and 7) extending away from the base 32 and slidingly mateable with the fluid inlet 92 of the sensor assembly 18. The nipple 94 has a flow passage 96. The second connector 40 is supported by the top side 58 of the base 32 and defines a first fluid outlet. The top side 58 of the base 32 may have a first opening 98 (FIG. 4) configured to receive the second connector 40. As best shown in FIG. 7, the second end 76 of the first tube 34 is connected to the second connector 40 to establish fluid communication with the flow passage 96 of the nipple 94. The second connector 40 may also include a seal member 100 (FIG. 7) positioned between the second end 76 of the first tube 34 and the nipple 94 to form a fluid tight seal. It should be appreciated the second connector 40 may be formed in any shape connectable to and disconnectable from the fluid inlet 92 of the sensor assembly 18.

The third connector 42 is adapted to mate with a fluid outlet (represented by the arrow 102 in FIG. 5) of the sensor assembly 18 in a way that fluid communication is established between the fluid outlet 102 of the sensor assembly 18 and the first end 78 of the second tube 36 and in a way that the third connector 42 can be quickly disconnected from the fluid outlet 102 of the sensor assembly 18.

In one embodiment, the third connector 42 has a nipple 104 (FIGS. 4, 5, and 7) extending away from the base 32 and slidingly mateable with the fluid outlet 102 of the sensor assembly 18. The nipple 104 has a flow passage 106 (FIG. 7). The third connector 42 is supported by the top side 58 of the base 32 and defines a second fluid inlet. The top side 58 of the base 32 may have a second opening 108 (FIG. 4) configured to receive the third connector 42. The first end 78 of the second tube 36 is connected to the third connector 42 to establish fluid communication with the flow passage 106 of the nipple 104. The third connector 42 may also include a seal member 110 (FIG. 7) positioned between the first end 78 of the second tube 36 and the nipple 104 to form a fluid tight seal. It should be appreciated the second third connector 42 may be formed in any shape connectable to and disconnectable from the fluid outlet 102 of the sensor assembly 18.

Because both the second connector 40 and the third connector 42 interface with the sensor assembly 18, the second connector 40 and the third connector 42 may be arranged in a side-by-side relationship to facilitate simultaneous connection with the sensor assembly 18 and simultaneous disconnection from the sensor assembly 18. The top side 58 of the base 32 may include a recess 112. The recess 112 is mateable with a male portion (not shown) of the sensor assembly 18. The nipple 94 of the second connector 40 and the nipple 104 of the third connector 42 may be secured to the base 32 with a retainer 114 (FIGS. 4 and 7). The retainer 114 may be connected to the base 32 in a suitable manner, such as with tabs, press fit, and/or fasteners and configured to define a recess 116 (FIGS. 3 and 7) to matingly engage the male portion of the sensor assembly 18.

Referring to FIGS. 4, 5, and 8, the fourth connector 44 is adapted to mate with a fluid inlet 116 of the waste conduit 30 (FIG. 5) in a way that fluid communication is established between the second end 80 of the second tube 36 and the fluid inlet 116 of the sensor assembly 18 and in a way that the fourth connector 44 can be quickly disconnected from the fluid inlet 116 of the waste conduit 30.

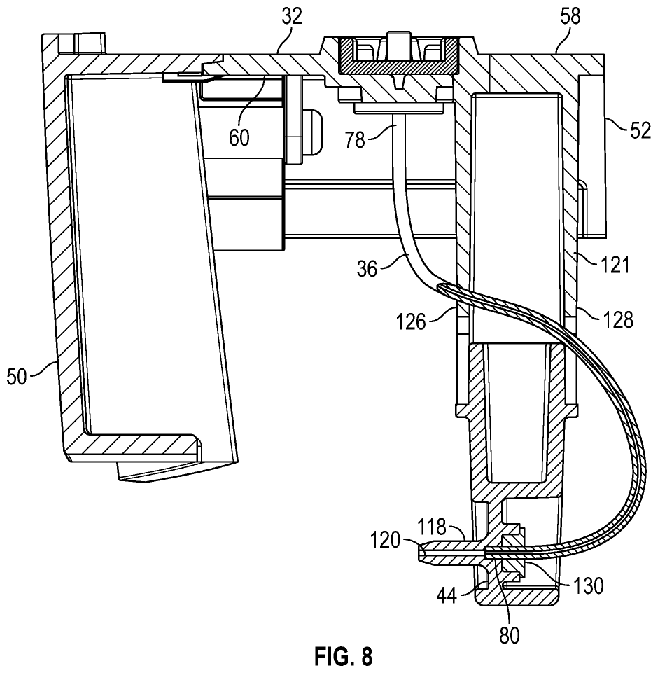

In one embodiment, the fourth connector 44 has a nipple 118 extending away from the base 32 and slidingly mateable with the fluid inlet 116 of the waste conduit 30. The nipple 118 has a flow passage 120 (FIG. 8). The bottom side 60 of the base 32 has a downwardly extending arm 121 with a proximal end 122 and a distal end 124. The fourth connector 44 is supported by the arm 121 adjacent the distal end 124 thereof. The arm 121 has a front side 126 and a rear side 128. The second tube 36 extends from the top side 58 of the base 32, downwardly through the arm 121 from the front side 126 to the rear side 128, and back through the arm 121 from the rear side 128 to the front side 130.

The fourth connector 44 extends from the distal end 124 of the arm 121 and defines a second fluid outlet. The second end 80 of the second tube 36 is connected to the fourth connector 44 to establish fluid communication with the flow passage 120 of the nipple 118. The fourth connector 44 may also include a seal member 130 positioned between the second end 80 of the second tube 36 and the nipple 118 to form a fluid tight seal. It should be appreciated the fourth connector 42 may be formed in any shape connectable to and disconnectable from the fluid inlet 116 of the waste conduit 30.

Each of the first connector 38, the second connector 40, the third connector 42, and the fourth connector 44 has been illustrated as being separate components from the base 32. It will be appreciated, however, that any number of the connectors 38-44 may be formed as a part of the base during the manufacturing process, such as by a suitable molding process.

In use, the fluidic tubing assembly 16 is inserted into and secured within the enclosure 12. The first connector 38 is connected to the fluid outlet 82 of the sample receiving assembly 14. In one embodiment, the sample receiving assembly 14 is moved axially into engagement with the first connector 38 either manually or mechanically. The second connector 40 and the third connector 42 are connected to fluid inlet 92 and the fluid outlet 102 of the sensor assembly 18, respectively. In one embodiment, the sensor assembly 18 is moved axially as a unit into engagement with the second connector 40 and the third connector 42 either manually or mechanically. The fourth connector 44 is connected to the fluid inlet 116 of the waste conduit 30. In one embodiment, the reagent assembly 20 is moved axially as a unit into engagement with the fourth connector 44 either manually or mechanically. It should be appreciated that the order of connection may be varied.

After a predetermined number of tests or upon determining the fluidic tubing assembly 16 has a blockage, the fluidic tubing assembly 16 may be removed from the enclosure 12 as a unit by disconnecting the first connector 38 from the fluid outlet 82 of the sample receiving assembly 14. Removal of the modular fluidic tubing assembly 16 effectuates a disconnection of the tubing between the sample receiving assembly 14, the sensor assembly 18, and the waste conduit 30. In one embodiment, the sample receiving assembly 14 may be moved axially away from the first connector 38 and removed from the enclosure 12. The second connector 40 and the third connector 42 are disconnected from the fluid inlet 92 and the fluid outlet 102 of the sensor assembly 18, respectively. In one embodiment, the sensor assembly 18 is disengaged from the second and third connectors 40 and 42 by moving the sensor assembly 18 axially away from the second connector 40 and the third connector 42. The fourth connector 44 is disconnected from the fluid inlet 116 of the waste conduit 30. In one embodiment, the reagent assembly 20 is disengaged from the fourth connector 44 by moving the reagent assembly 20 axially away from the fourth connector 44. The fluidic tubing assembly 16 is then removed from the enclosure 12.

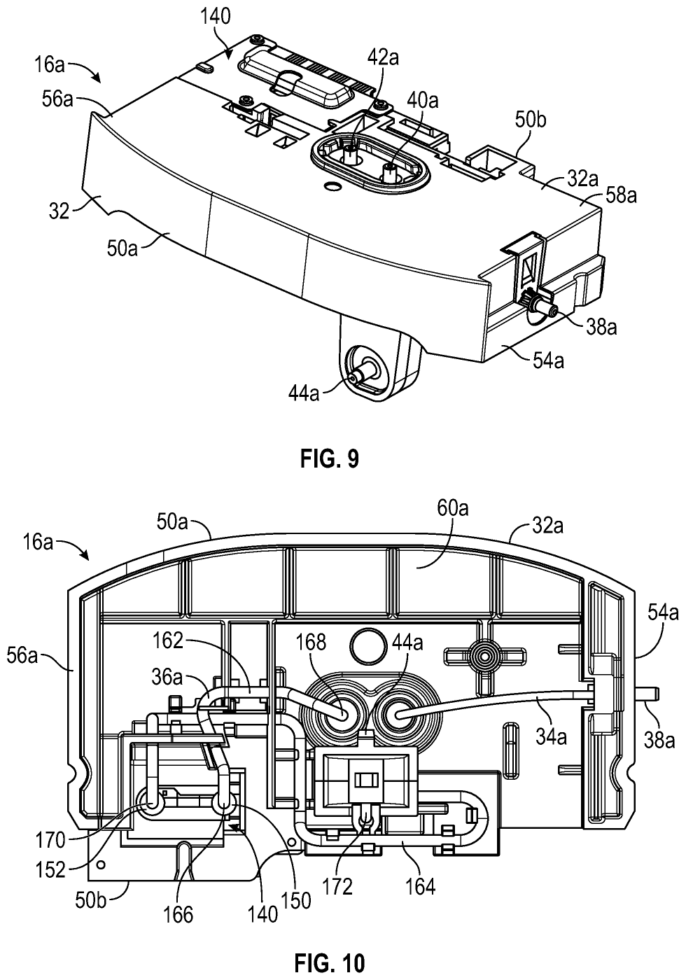

Referring now to FIGS. 9-12, another exemplary embodiment of a fluidic tubing assembly 16a is illustrated. The fluidic tubing assembly 16a is substantially similar to the fluidic tubing assembly 16, with the exception that the fluidic tubing assembly 16a includes a CO-oximetry optical cell 140. Like the fluidic tubing assembly 16, the fluidic tubing assembly 16a is a modular unit that provides fluid communication from the sample receiving assembly 14 to the sensor assembly 18 and from the sensor assembly 18 to the waste conduit 30 (FIG. 5) in fluid communication with the waste fluid collection member. The fluids tubing assembly 16a is configured to be positioned in the enclosure 12 of the blood analyzer 10 and broadly includes a base 32a, a first tube 34a, a second tube 36a, a first connector 38a, a second connector 40a, a third connector 42a, and a fourth connector 44a. The fluidic tubing assembly 16 is configured such that the first tube 34a and the second tube 36a connecting the various components of the blood analyzer 10 may be removably disconnected and/or replaced from the blood analyzer 10 without direct contact with the first tube 34a and the second tube 36a. Further, the removal of fluidic tubing assembly 16a permits for the simultaneous replacements of all fluidic tubing between the sample receiving assembly 14, the sensor assembly 18, and the reagent assembly 20.

The base 32a supports the others parts of the fluidic tubing assembly 16a including the CO-oximetry optical cell 140, so a fluid path is established between the components while allowing the fluidic tubing assembly 16a to be easily replaced. In a non-limiting example, the base 32a has a generally rectangular configuration with a front side 50a, a rear side 52a opposite the front side 50a, a first side 54a, a second side 56a opposite the first side 54a, a top side 58a, and a bottom side 60a opposite the top side 58a.

In one non-limiting embodiment, the base 32a supports the CO-oximetry optical cell 140. CO-oximetry is a spectroscopic or optical technique that is used to measure the amount of different Hemoglobin (Hb) species present in a blood sample, for example, Oxy-Hb, Deoxy-Hb, Met-Hb, Carboxy-Hb and Total-Hb. The CO-oximetry optical cell 140 may include a printed circuit board 142, a transducer 144, an optical cell 146, a cover 148, a first connector 150, and a second connector 152. The optical cell 146 may be formed of an upper transparent layer 154 and a lower transparent layer 156 cooperating to form a channel 158. The cover 148 has an opening 160 so a light source (not shown) incorporated as part of the blood analyzer 10 may be received by the fluid sample flowing through the channel 158. The first connector 150 and the second connector 152 are fluidically connected to the channel 158 to form an inlet and an outlet of the channel 158. An exemplary CO-oximetry optical cell is disclosed in PCT/US2021/029119, which is hereby incorporated herein by reference.

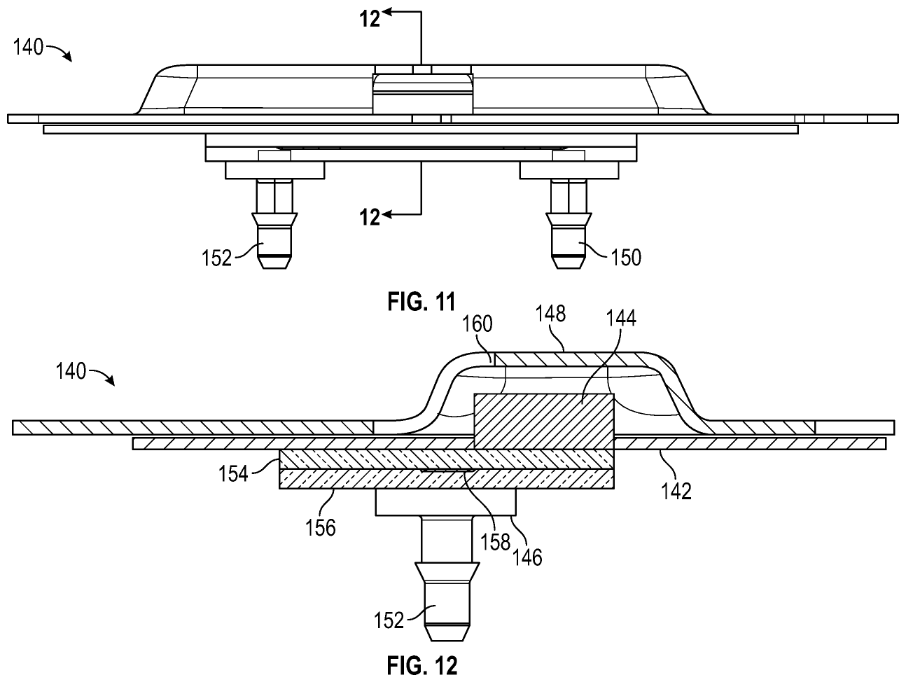

As shown in FIG. 10, the CO-oximetry optical cell 140 may be fluidically interposed in the second tube 36a between the third connector 42 and the fourth connector 44. To this end, the second tube 36a includes a first tube section 162 and a second tube section 164. The first connector 150 of the CO-oximetry optical cell 140 is adapted to mate with a second end 166 of the first tube section 162. A first end 168 of the first tube section 162 is connected to the fluid outlet (represented by the arrow 102 in FIG. 5) of the sensor assembly 18 as described above. The second connector 152 of the CO-oximetry optical cell 140 is adapted to mate with a first end 170 of the second tube section 164. A second end 172 of the second tube section 164 is connected to the fourth connector 44 as described above relative to the second end 80 of the second tube 36 to establish fluid communication with the flow passage 120 of the nipple 118. In one embodiment, the first connector 150 and the second connector 150 of the CO-oximetry optical cell 140 are in the form of nipples (FIGS. 11 and 12).

In another embodiment, the CO-oximetry optical cell 140 may be fluidically interposed in the first tube 34a between the first connector 38 and the second connector 40. In this instance, the first tube 34a may be formed of one or more sections.

In use, the fluidic tubing assembly 16a is inserted into and secured within the enclosure 12 the same as described above for the fluidic tubing assembly 16. After a predetermined number of tests or upon determining the fluidic tubing assembly 16a has a blockage, the fluidic tubing assembly 16a may be removed from the enclosure 12 as a unit similar to manner described above for the fluidic tubing assembly 16.

From the above description, it is clear that the inventive concept(s) disclosed herein is well adapted to carry out the objects and to attain the advantages mentioned herein as well as those inherent in the inventive concept disclosed herein. While exemplary embodiments of the inventive concept disclosed herein have been described for purposes of this disclosure, it will be understood that numerous changes may be made which will readily suggest themselves to those skilled in the art and which are accomplished without departing from the scope of the inventive concept disclosed herein and defined by the appended claims.

The following is a list of non-limiting illustrative embodiments of the inventive concepts disclosed herein:

An illustrative fluidic tubing assembly for a blood analyzer having a housing supporting a fluid sample assembly, a sensor assembly, and a fluid waste assembly, the fluidic tubing assembly comprising:
a base positionable in the housing of the blood analyzer;
a plurality of connectors extending from the base so each of the connectors is removably connectable to at least one of the fluid sample assembly, the sensor assembly, and the fluid waste assembly; and
a plurality of tubes, each of the tubes extending from one of the connectors to another one of the connectors to establish fluid communication from one of the connectors to the other connector,
wherein fluid communication is established between the fluid sample assembly, the sensor assembly, and the fluid waste assembly through the fluidic tubing assembly when the fluidic tubing assembly is positioned in the housing.

The illustrative fluidic tubing assembly of any one of the preceding illustrative embodiments further comprising a CO-oximetry optical cell having a channel fluidically interposed between at least two of the connectors.

An illustrative fluidic tubing assembly for a blood analyzer, comprising:
a base connectable to the blood analyzer and having a front side, a rear side opposite the front side, a first side, a second side opposite the first side, a top side, and a bottom side opposite the top side;
a first tube having a first end and a second end;
a second tube having a first end and a second end;
a first connector supported by the first side of the base and defining a first fluid inlet, the first end of the first tube connected to the first connector;
a second connector supported by the top side of the base and defining a first fluid outlet, the second end of the first tube connected to the second connector;
a third connector supported by the top side of the base and defining a second fluid inlet, the first end of the second tube connected to the third connector; and
a fourth connector supported by the bottom side of the base and defining a second fluid outlet, the second end of the second tube connected to the fourth connector.

The illustrative fluidic tubing assembly of any one of the preceding illustrative embodiments wherein the first connector has a nipple extending away from the base.

The illustrative fluidic tubing assembly of any one of the preceding illustrative embodiments wherein the second connector has a nipple extending away from the base.

The illustrative fluidic tubing assembly of any one of the preceding illustrative embodiments wherein the third connector has a nipple extending away from the base.

The illustrative fluidic tubing assembly of any one of the preceding illustrative embodiments wherein the fourth connector has a nipple extending away from the base.

The illustrative fluidic tubing assembly of any one of the preceding illustrative embodiments wherein each of the first connector, the second connector, the third connector, and the fourth connector has a nipple extending away from the base.

The illustrative fluidic tubing assembly of any one of the preceding illustrative embodiments wherein the top side of the base has a recess, and wherein the second connector and the third connector are positioned within the recess.

The fluidic tubing assembly of any one of the preceding illustrative embodiments wherein each of the second connector and the third connector has a nipple extending away from the base.

The illustrative fluidic tubing assembly of any one of the preceding illustrative embodiments wherein the bottom side of the base has a downwardly extending arm with a proximal end and a distal end, and wherein the fourth connector is supported by the arm adjacent the distal end thereof.

The illustrative fluidic tubing assembly of any one of the preceding illustrative embodiments wherein the fourth connector has a nipple extending away from the arm.

The illustrative fluidic tubing assembly of any one of the preceding illustrative embodiments wherein the arm has a front side and a rear side, and wherein the second tube extends from the top side of the base, downwardly through the arm from the front side to the rear side, and back through the arm from the rear side to the front side.

The illustrative fluidic tubing assembly of any one of the preceding illustrative embodiments wherein first side of the base has a first rail extending from the rear side toward the front side, and wherein the second side of the base has a second rail extending from the rear side toward the front side.

The illustrative fluidic tubing assembly of any one of the preceding illustrative embodiments further comprising a CO-oximetry optical cell having a channel fluidically interposed between at least one of the first connector and the second connector and the third connector and the fourth connector tube.

The illustrative fluidic tubing assembly of any one of the preceding illustrative embodiments further comprising a CO-oximetry optical cell having a channel fluidically interposed between the third connector and the fourth connector.

An illustrative blood analyzer, comprising
a housing;
a sensor assembly positioned in the housing and having a fluid inlet and a fluid outlet;
a reagent assembly having a fluid outlet;
a sample receiving assembly having a sample probe with a fluid inlet and a fluid outlet, the sample probe movable between a first position wherein the sample probe is in fluid communication with the fluid outlet of the reagent assembly and a second position wherein the sample probe is connectable to a sample transport container;
a fluidic tubing assembly, comprising
   a base positioned in the housing and having a front side, a rear side opposite the front side, a first side, a second side opposite the first side, a top side, and a bottom side opposite the top side;
   a first tube having a first end and a second end;
   a second tube having a first end and a second end;
   a first connector supported by the first side of the base and defining a first fluid inlet, the first end of the first tube connected to the first connector, the first connector connected to the fluid outlet of the sample receiving assembly;
   a second connector supported by the top side of the base and defining a first fluid outlet, the second end of the first tube connected to the second connector, the second connector connected to the fluid inlet of the sensor assembly;
   a third connector supported by the top side of the base and defining a second fluid inlet, the first end of the second tube connected to the third connector, the third connector connected to the fluid outlet of the sensor assembly; and
   a fourth connector supported by the bottom side of the base and defining a second fluid outlet, the second end of the second tube connected to the fourth connector, the fourth connector connected to a waste conduit.

The illustrative blood analyzer of any one of the preceding illustrative embodiments wherein the fluidic tubing assembly comprises a CO-oximetry optical cell having a channel in fluid communication with the sensor assembly, the fluid waste assembly, and the sample receiving assembly.

The illustrative blood analyzer of any one of the preceding illustrative embodiments, wherein the first connector has a nipple extending away from the base.

The illustrative blood analyzer of any one of the preceding illustrative embodiments wherein the second connector has a nipple extending away from the base.

The illustrative blood analyzer of any one of the preceding illustrative embodiments wherein the third connector has a nipple extending away from the base.

The illustrative blood analyzer of any one of the preceding illustrative embodiments wherein the fourth connector has a nipple extending away from the base.

The illustrative blood analyzer of any one of the preceding illustrative embodiments wherein each of the first connector, the second connector, the third connector, and the fourth connector has a nipple extending away from the base.

The illustrative blood analyzer of any one of the preceding illustrative embodiments wherein the top side of the base has a recess, and wherein the second connector and the third connector are positioned within the recess.

The illustrative blood analyzer of any one of the preceding illustrative embodiments wherein each of the second connector and the third connector has a nipple extending away from the base.

The illustrative blood analyzer of any one of the preceding illustrative embodiments wherein the bottom side of the base has a downwardly extending arm with a proximal end and a distal end, and wherein the fourth connector is supported by the arm adjacent the distal end thereof.

The illustrative blood analyzer of any one of the preceding illustrative embodiments wherein the fourth connector has a nipple extending away from the arm.

The illustrative blood analyzer of any one of the preceding illustrative embodiments wherein the arm has a front side and a rear side, and wherein the second tube extends from the top side of the base, downwardly through the arm from the front side to the rear side, and back through the arm from the rear side to the front side.

The illustrative blood analyzer of any one of the preceding illustrative embodiments wherein first side of the base has a first rail extending from the rear side toward the front side, and wherein the second side of the base has a second rail extending from the rear side toward the front side.

An illustrative method of providing fluid passage in a blood analyzer, the blood analyzer having a housing, a sample receiving assembly, a sensor assembly, and a fluid waste assembly, the method comprising:
obtaining a fluidic tubing assembly for a blood analyzer, the fluidic tubing assembly comprising:
a base connectable to the blood analyzer and having a front side, a rear side opposite the front side, a first side, a second side opposite the first side, a top side, and a bottom side opposite the top side;
a first tube having a first end and a second end;
a second tube having a first end and a second end
a first connector supported by the first side of the base and defining a first fluid inlet, the first end of the first tube connected to the first connector;
a second connector supported by the top side of the base and defining a first fluid outlet, the second end of the first tube connected to the second connector;
a third connector supported by the top side of the base and defining a second fluid inlet, the first end of the second tube connected to the third connector; and
a fourth connector supported by the bottom side of the base and defining a second fluid outlet, the second end of the second tube connected to the fourth connector; and
positioning the fluidic tubing assembly in the housing;
connecting the first connector to the fluid supply assembly;
connecting the second connector and the third connector to the sensor assembly; and
connecting the fourth connector to the fluid waste assembly.

The illustrative method of any one of the preceding illustrative embodiments wherein the fluidic tubing assembly is a first fluidic tubing assembly, and wherein the method further comprises:
disconnecting the first connector from the fluid supply assembly;
disconnecting the second connector and the third connector from the sensor assembly;
disconnecting the fourth connector from the fluid waste assembly;
removing the first fluid tubing assembly from the housing; and
obtaining a second fluidic tubing assembly of like construction to the first fluidic tubing assembly;
positioning the second fluidic tubing assembly in the housing; and
connecting the second fluidic tubing assembly to the fluid supply assembly to the sensor assembly, and to the fluid waste assembly.

What is claimed:

1. A fluidic tubing assembly for a blood analyzer having a housing supporting a fluid sample assembly, a sensor assembly, and a fluid waste assembly, the fluidic tubing assembly comprising:
a base positionable in the housing of the blood analyzer;
a plurality of connectors extending from the base so each of the connectors is removably connectable to at least one of the fluid sample assembly, the sensor assembly, and the fluid waste assembly; and
a plurality of tubes, each of the tubes extending from one of the connectors to another one of the connectors to establish fluid communication from one of the connectors to the other connector,
wherein fluid communication is established between the fluid sample assembly, the sensor assembly, and the fluid waste assembly through the fluidic tubing assembly when the fluidic tubing assembly is positioned in the housing.

2. The fluidic tubing assembly of claim 1, further comprising a CO-oximetry optical cell having a channel fluidically interposed between at least two of the connectors.

3. The fluidic tubing assembly of claim 1, wherein the base has a front side, a rear side opposite the front side, a first side, a second side opposite the first side, a top side, and a bottom side opposite the top side, wherein the plurality of tubes comprises a first tube having a first end and a second end, and a second tube having a first end and a second end, and wherein the plurality of connectors comprises:
a first connector supported by the first side of the base and defining a first fluid inlet, the first end of the first tube connected to the first connector;
a second connector supported by the top side of the base and defining a first fluid outlet, the second end of the first tube connected to the second connector;
a third connector supported by the top side of the base and defining a second fluid inlet, the first end of the second tube connected to the third connector; and
a fourth connector supported by the bottom side of the base and defining a second fluid outlet, the second end of the second tube connected to the fourth connector.

4. The fluidic tubing assembly of claim 3, wherein the first connector has a nipple extending away from the base.

5. The fluidic tubing assembly of claim 3, wherein the second connector has a nipple extending away from the base.

6. The fluidic tubing assembly of claim 3, wherein the third connector has a nipple extending away from the base.

7. The fluidic tubing assembly of claim 3, wherein the fourth connector has a nipple extending away from the base.

8. The fluidic tubing assembly of claim 3, wherein each of the first connector, the second connector, the third connector, and the fourth connector has a nipple extending away from the base.

9. The fluidic tubing assembly of claim 3, wherein the top side of the base has a recess, and wherein the second connector and the third connector are positioned within the recess.

10. The fluid tubing assembly of claim 9, wherein each of the second connector and the third connector has a nipple extending away from the base.

11. The fluidic tubing assembly of claim 3, wherein the bottom side of the base has a downwardly extending arm with a proximal end and a distal end, and wherein the fourth connector is supported by the arm adjacent the distal end thereof.

12. The fluidic tubing assembly of claim 11, wherein the fourth connector has a nipple extending away from the arm.

13. The fluidic tubing assembly of claim 11, wherein the arm has a front side and a rear side, and wherein the second tube extends from the top side of the base, downwardly through the arm from the front side to the rear side, and back through the arm from the rear side to the front side.

14. The fluidic tubing assembly of claim 3, wherein first side of the base has a first rail extending from the rear side toward the front side, and wherein the second side of the base has a second rail extending from the rear side toward the front side.

15. The fluidic tubing assembly of claim 3, further comprising a CO-oximetry optical cell having a channel fluidically interposed between at least one of the first connector and the second connector and the third connector and the fourth connector tube.

16. The fluidic tubing assembly of claim 3, further comprising a CO-oximetry optical cell having a channel fluidically interposed between the third connector and the fourth connector.

17. A blood analyzer, comprising
a housing supporting:
a sensor assembly having a fluid inlet and a fluid outlet;
a fluid waste assembly;
a sample receiving assembly having a sample probe with a fluid inlet and a fluid outlet; and
a fluidic tubing assembly removably connected to the sensor assembly, the fluid waste assembly, and the sample receiving assembly so fluid communication is established between the sensor assembly, the fluid waste assembly, and the sample receiving assembly.

18. The blood analyzer of claim 17, wherein the sample probe is movable between a first position wherein the sample probe is in fluid communication with the fluid outlet of the reagent assembly and a second position wherein the sample probe is connectable to a sample transport container.

19. The blood analyzer of claim 17, wherein the fluidic tubing assembly comprises a CO-oximetry optical cell having a channel in fluid communication with the sensor assembly, the fluid waste assembly, and the sample receiving assembly.

20. The blood analyzer of claim 17, wherein the fluidic tubing assembly comprises:
a base having a front side, a rear side opposite the front side, a first side, a second side opposite the first side, a top side, and a bottom side opposite the top side;
a first tube having a first end and a second end;
a second tube having a first end and a second end;
a first connector supported by the first side of the base and defining a first fluid inlet, the first end of the first tube connected to the first connector, the first connector connected to the fluid outlet of the sample receiving assembly;
a second connector supported by the top side of the base and defining a first fluid outlet, the second end of the first tube connected to the second connector, the second connector connected to the fluid inlet of the sensor assembly;
a third connector supported by the top side of the base and defining a second fluid inlet, the first end of the second tube connected to the third connector, the third connector connected to the fluid outlet of the sensor assembly; and
a fourth connector supported by the bottom side of the base and defining a second fluid outlet, the second end of the second tube connected to the fourth connector, the fourth connector connected to a waste conduit.

21. The blood analyzer of claim 20, wherein the fluidic tubing assembly comprises a CO-oximetry optical cell having a channel fluidically interposed between at least one of the first connector and the second connector and the third connector and the fourth connector tube.

22. The blood analyzer of claim 20, wherein the fluidic tubing assembly further comprises a CO-oximetry optical cell having a channel fluidically interposed between the third connector and the fourth connector.

23. The blood analyzer of claim 17, wherein the first connector has a nipple extending away from the base.

24. The blood analyzer of claim 17, wherein the second connector has a nipple extending away from the base.

25. The blood analyzer of claim 17, wherein the third connector has a nipple extending away from the base.

26. The blood analyzer of claim 17, wherein the fourth connector has a nipple extending away from the base.

27. The blood analyzer of claim 17, wherein each of the first connector, the second connector, the third connector, and the fourth connector has a nipple extending away from the base.

28. The blood analyzer of claim 17, wherein the top side of the base has a recess, and wherein the second connector and the third connector are positioned within the recess.

29. The blood analyzer of claim 28, wherein each of the second connector and the third connector has a nipple extending away from the base.

30. The blood analyzer of claim 17, wherein the bottom side of the base has a downwardly extending arm with a proximal end and a distal end, and wherein the fourth connector is supported by the arm adjacent the distal end thereof.

31. The blood analyzer of claim 30, wherein the fourth connector has a nipple extending away from the arm.

32. The blood analyzer of claim 30, wherein the arm has a front side and a rear side, and wherein the second tube extends from the top side of the base, downwardly through the arm from the front side to the rear side, and back through the arm from the rear side to the front side.

33. The blood analyzer of claim 17, wherein first side of the base has a first rail extending from the rear side toward the front side, and wherein the second side of the base has a second rail extending from the rear side toward the front side.

34. A method of operating a blood analyzer, the blood analyzer having a sensor assembly, a sample receiving assembly, and a fluid waste assembly, comprising the steps of:
(a) obtaining a modular, removable fluidic tubing assembly for establishing fluid communication between at least the sample receiving assembly, the sensor assembly, and the fluid waste assembly; and
(b) inserting the fluidic tubing assembly within the blood gas analyzer and establishing fluid communication between the sample receiving assembly, the sensor assembly, and the fluid waste assembly via the fluidic tubing assembly.

35. The method of claim 34, wherein the step of obtaining the modular, removeable fluidic tubing assembly further comprises the modular, removable fluidic tubing assembly having a CO-oximetry optical cell, and wherein the step of inserting the fluidic tubing assembly further comprises establishing fluid communication between the sample receiving assembly, the sensor assembly, and the fluid waste assembly via the CO-oximetry optical cell.

36. A method of operating a blood gas analyzer, the blood analyzer having a modular, removable fluidic tubing assembly, the method comprising the steps of:
(a) providing a sensor assembly, a sample receiving assembly, and a fluid waste assembly, the sensor assembly, the sample receiving assembly, and the fluid waste assembly being in non-fluid communication;
(b) connecting the fluidic tubing assembly to the sensor assembly,
(c) connecting the fluidic tubing assembly to the fluid waste assembly; and
(d) connecting the fluidic tubing assembly to the sample receiving assembly,
wherein the steps of (b) through (d) establishes fluid communication between the sensor assembly, the sample receiving assembly, and the fluid waste assembly.

37. The method of claim 36, wherein the fluidic tubing assembly has a CO-oximetry optical cell, and wherein the steps of (b) through (d) establishes fluid communication between the sample receiving assembly, the sensor assembly, and the fluid waste assembly via the CO-oximetry optical cell.

38. A method of providing fluid passage in a blood analyzer, the blood analyzer having a housing, a sample receiving assembly, a sensor assembly, and a fluid waste assembly, the method comprising:
obtaining a fluidic tubing assembly for a blood analyzer, the fluidic tubing assembly comprising:
a base connectable to the blood analyzer and having a front side, a rear side opposite the front side, a first side, a second side opposite the first side, a top side, and a bottom side opposite the top side;
a first tube having a first end and a second end;
a second tube having a first end and a second end;
a first connector supported by the first side of the base and defining a first fluid inlet, the first end of the first tube connected to the first connector;
a second connector supported by the top side of the base and defining a first fluid outlet, the second end of the first tube connected to the second connector;
a third connector supported by the top side of the base and defining a second fluid inlet, the first end of the second tube connected to the third connector; and
a fourth connector supported by the bottom side of the base and defining a second fluid outlet, the second end of the second tube connected to the fourth connector; and
positioning the fluidic tubing assembly in the housing;
connecting the first connector to the fluid supply assembly;
connecting the second connector and the third connector to the sensor assembly; and
connecting the fourth connector to the fluid waste assembly.

39. The method of claim 38, wherein the fluidic tubing assembly is a first fluidic tubing assembly, and wherein the method further comprises:
disconnecting the first connector from the fluid supply assembly;
disconnecting the second connector and the third connector from the sensor assembly;
disconnecting the fourth connector from the fluid waste assembly;
removing the first fluid tubing assembly from the housing; and
obtaining a second fluidic tubing assembly of like construction to the first fluidic tubing assembly;
positioning the second fluidic tubing assembly in the housing; and
connecting the second fluidic tubing assembly to the fluid supply assembly to the sensor assembly, and to the fluid waste assembly.
